(19) United States
(12) Patent Application Publication
(10) Pub. No.: US 2026/0166226 A1
(43) Pub. Date: Jun. 18, 2026

(54) IDENTIFYING POTENTIAL ANALYTE SENSOR FAULTS THROUGH INTERACTION WITH AMD SYSTEMS

(71) Applicant: INSULET CORPORATION, Acton, MA (US)

(72) Inventors: Joon Bok LEE, Acton, MA (US); Dirceu CAVENDISH, Escondido, CA (US); Jason O’CONNOR, Acton, MA (US); Yibin ZHENG, Hartland, WI (US)

(21) Appl. No.: 19/419,900

(22) Filed: Dec. 15, 2025

Related U.S. Application Data
(60) Provisional application No. 63/734,559, filed on Dec. 16, 2024.

(51) Int. Cl. A61M 5/172; A61B 5/145; A61M 5/142

(52) U.S. Cl. CPC A61M 5/1723; A61B 5/14532; A61M 5/14244; A61B 2560/0276

(57) ABSTRACT

Presented is a medicament delivery system including a controller, a wearable medication delivery device, an analyte sensor, and other devices. In an example, a receiver is operable to receive glucose measurement values from the analyte sensor. A number of devices of the medicament delivery system may include a processor operable to execute programming code and to identify anomalous readings among the received glucose measurement values. The identified anomalous reading may, for example, be based on the received glucose measurement values violating a measurement change threshold indicative of abnormal sensor behavior. The processor may be operable to, in response to identifying the anomalous reading, revert a medicament delivery setting to a default safety basal delivery setting and/or generate a notification, an inquiry, and/or an alert for presentation to a user.

### CROSS-REFERENCE TO RELATED APPLICATIONS

This application claims the benefit of and priority to U.S. Provisional Patent Application No. 63/734,559 filed on Dec. 16, 2024, which is incorporated herein by reference in its entirety.

### BACKGROUND

In some drug delivery systems, there are sensor independent methods for adjusting safety constraints that may adjust drug delivery. However, these sensor independent methods do not address or indicate potential problems with the sensor. For example, the sensor independent methods may review a total amount of medicament delivered, make a decision about how much should have been delivered, and, based on that decision, may adjust the basal delivery dosages, all without any input from a glucose sensor.

Glucose sensors are typically considered a source of truth when utilized with automated medicament delivery (AMD) systems, and any errors in AMD actions are usually validated against the glucose readings from the glucose sensors. However, this architecture can have significant vulnerabilities to any faults that may arise in the glucose sensors. While glucose sensor fault mitigation responses may be implemented in the AMD system, the fault mitigation responses may not be optimal for a user. For example, AMD systems typically may either rely on error messages from error detection algorithms executed by the respective sensor or by the AMD algorithm. The AMD algorithm may have an error detection algorithm that reacts to a user's insulin delivery history. However, it takes time to accumulate enough data in the user's insulin delivery history to make a determination as to whether there may be a measurement issue with a sensor.

In response to the detection of an error, the AMD system may implement constraints on medicament, such as insulin delivery, which are independent of analyte readings, these mitigations typically ensure safe, but not necessarily optimal, responses to such analyte sensor faults.

Hence, there is an opportunity for improvement in responding to sensor faults by determining potential sensor faults early in the execution of the AMD algorithm processes and producing recommended patient interactions before the system defaults to less optimal, safe behaviors.

### BRIEF SUMMARY

In one aspect, a medicament delivery system includes a receiver operable to receive glucose measurement values from an analyte sensor. The medicament delivery system also includes a processor operable to execute programming code and operable to identify anomalous readings among the received glucose measurement values, where the identified anomalous readings are based on the received glucose measurement values violating a measurement change threshold indicative of abnormal sensor behavior, and in response to identifying the anomalous readings, revert a medicament delivery setting to a default safety basal delivery setting and/or generate an alert for presentation to a user.

In another aspect, a controller includes memory storing programming instructions. The controller also includes a processor coupled to the memory and configured to identify anomalous measurements indicative of abnormal sensor behavior among the received glucose measurement values, where the identified anomalous measurements are identified by: comparing presently received glucose measurement values to previously received glucose measurement values, determining whether a result of the comparing indicates that the presently received glucose measurement values violated a measurement change threshold, and identifying the received glucose measurements and previously received glucose measurements as anomalous measurements. The controller also includes, in response to identifying the anomalous measurements, reverting a medicament delivery setting to a default safety delivery setting.

In one aspect, a controller includes a memory storing programming instructions. The controller also includes a processor coupled to the memory and configured to estimate future sensor measurement values for future times based on previous sensor measurement values, where each future sensor measurement value of the estimated future analyte values is estimated for a different respective future time, determining a difference between an actual analyte measurement value obtained at each respective future time and the estimated future sensor measurement value, evaluate the difference with respect to previously determined differences, and based on the result of the evaluation exceeding a difference threshold value, generate a notification signal indicating a sensor error.

### BRIEF DESCRIPTION OF THE DRAWINGS

To easily identify the discussion of any particular element or act, the most significant digit or digits in a reference number refer to the figure number in which that element is first introduced.

FIG. 1 illustrates an exemplary process for identifying a potential faulty analyte sensor according to the disclosed subject matter.

FIG. 2 illustrates a further exemplary process for identifying a potential faulty analyte sensor according to the disclosed subject matter.

FIG. 3 illustrates an exemplary medicament system operable to implement the examples disclosed herein.

### DETAILED DESCRIPTION

At times, an automated medicament delivery algorithm may reevaluate parameter settings based on the amount of medicament delivered over a set period of time (i.e., a medicament delivery history). Instead of waiting for a user medicament delivery history to be accumulated and serve as an indicator of a sensor issue, the lack of sensor readings that are within physiological thresholds, present as atypical sensor readings, or the like (e.g., anomalous reading indicative of abnormal sensor behavior) may be used to cause an automated medicament delivery (AMD) algorithm as described herein to respond to the anomalous reading. The AMD algorithm may identify an issue with the sensor and generate a response (e.g., modification to AMD algorithm parameters) optimized for the user as well as generate notifications or alerts for the user or health care provider.

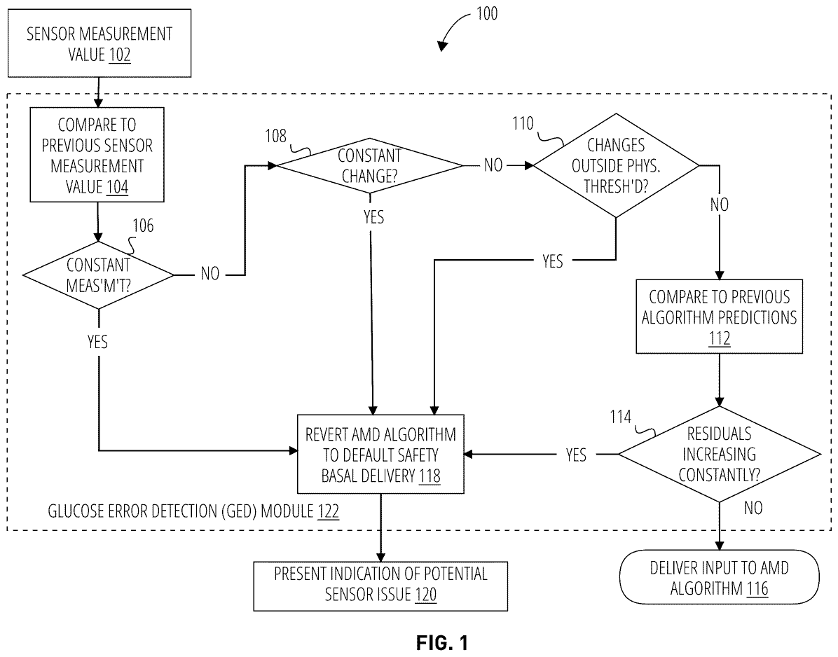

FIG. 1 illustrates an example process 100 for determining whether an analyte sensor is operating properly. A processor may execute process 100 by using software, hardware, firmware, or a combination thereof. The analyte sensor may be considered to be operating properly when abnormal behavior is not identified. Additionally, when abnormal behavior of the analyte sensor is identified, the system may respond by adjusting operation of the AMD algorithm to provide user-optimized levels of medicament delivery. The user-optimized levels of medicament delivery are based on more specific AMD settings that are derived as described in more detail below.

In response to the identified abnormal analyte sensor behavior, the processor may, for example, be operable to set a default safety delivery parameter of the AMD algorithm that causes the medicament delivery device to deliver a user-optimized and safe dosage of medicament. Additionally, the system may react to provide an indication on an output device that the analyte sensor should be checked for proper operation.

In an example, the process 100 may be implemented by a medicament delivery system that includes a receiver operable to receive glucose measurement values from an analyte sensor. The medicament delivery system also includes a processor operable to execute programming code, such as a glucose error detection (GED) module 122. The processor, when executing the glucose error detection (GED) module 122, may be operable to identify anomalous readings indicative of abnormal sensor behavior among the received glucose measurement values. In an example, the glucose error detection (GED) module 122 may be implemented in combination with an AMD algorithm. Alternatively, the AMD algorithm may control implementation of the glucose error detection (GED) module 122. For example, the glucose error detection (GED) module 122 may be a routine executed by the AMD algorithm.

At a high level, the glucose error detection (GED) module 122 is operable to identify an anomalous measurement indicative of abnormal sensor behavior among the glucose measurement values received from a sensor. When identifying readings indicative of abnormal sensor behavior during the execution of the GED module, the processor is further operable to determine whether the glucose measurement values vary more than a physiological threshold over a sensor check sample period. In an example, the sensor check sample period may be 1.5, 2 or 3 hours, or may be a range between 1 hour and 2 hours, 1 to 3 hours, or the like. Additionally, or alternatively, the sensor check sample period may be measured in operational cycles of the system or a sensor, for example, 12 sensor operational cycles, 24 sensor operational cycles, 18 system operational cycles, 24 system operational cycles, or any number of cycles falling between 12 sensor/system operational cycles and 24 sensor/system operational cycles, or the like. At times, a duration of the operational cycle of a sensor and the system may be the same. In some examples, a sensor operational cycle is 5 minutes meaning the sensor provides a sensor measurement value to the processor and the AMD algorithm at 5 minute intervals. A system operational cycle includes receipt of the sensor measurement value and additional functions, such as delivery of medicament, setting of AMD algorithm parameters, system status checks, or the like. The physiological threshold may be referred to as a “physiological variation threshold” in this example (e.g., an amount of variation expected to be seen in sensor measurement values, such as glucose measurement values). For example, a physiological variation threshold for a glucose measurement value may be a variation of plus or minus 5 mg/dL over a sensor check sample period.

An anomalous reading, which is equivalent to an anomalous measurement, may be any sensor reading that is erroneous due to at least one of a constant measurement, a constant change, or a change outside a physiological threshold (each are described further below with respect to specific evaluations of each). For example, the identified anomalous measurement may be identified based on the received glucose measurement values violating a measurement change threshold that includes a physiological threshold as well as a physiological variation threshold, a change variation threshold, a combination of thresholds, or the like.

In addition, or alternatively, the system may identify sensor measurement values as anomalous measurements when the sensor measurement value violates the measurement change threshold with respect to one or more previously received glucose measurement values. The previously received glucose measurement values may be glucose measurement values received earlier than or prior to presently received glucose measurement values. The “presently received glucose measurement value” is the glucose measurement value most recently received by the system. An “immediate past glucose measurement value” is the glucose measurement value received immediately prior to another glucose measurement value, with no other glucose measurement value received in the between the two glucose measurement values. In response to identifying the anomalous measurements, the processor may cause the AMD algorithm to revert a medicament delivery setting to a default safety delivery setting, and/or generate an alert for presentation to a user. The medicament delivery setting may be a setting of the AMD algorithm related to a dosage of medicament to be delivered by the medicament delivery system at least in a next operational cycle, or a number of cycles for a predetermined period of time.

In process 100, the glucose error detection (GED) module 122 may receive a present sensor measurement value 102. The sensor measurement value 102 may be an output from an analyte sensor, such as a continuous glucose monitor, a ketone sensor, or the like as shown in later examples.

The GED module may, for example, be operable to execute a step 104 that compares the sensor measurement value 102 to previous sensor measurement value(s). In the example, a previous analyte reading may be an analyte reading that is received prior to (or earlier than) a subsequent or a later but present sensor measurement value 102. In addition, the results of the comparison at 104 may be stored, so trends of the sensor measurement values may be determined. Further, multiple comparisons between a present sensor measurement value and earlier sensor measurement values may be made and used in the evaluations that follow in steps 106-114.

In one example, a user's true glucose concentrations vary in minute amounts even in cases where the user is otherwise completely at rest without any disturbances, as the human body is not a static system. Thus, if a glucose reading meets a pattern of constant readings far below the variability expected for a user without any disturbances, the system may alert the user as a potential mechanical or software fault of the sensor, and revert to safe insulin delivery.

The GED module may be operable to determine whether the amount of variability of the glucose measurement values is acceptable. Based on the result of the comparison, the GED module determines, at 106, based on the result of the comparison at 104 whether present sensor measurement value 102 is the same value as the previous (i.e., earlier) analyte reading. If the previous analyte reading value is the same as the sensor measurement value 102, this indicates that the readings (or measurements) from the analyte sensor(s) are a constant value. A sensor measurement value 102 may be considered a “same value” or a “constant value” when the value of a current analyte measurement value is within ±0.0-0.75 mg/dL of a previous analyte measurement value, or the like. In an example, the processor when executing programming instructions of the GED module for the decision at 106 may be operable to evaluate a number of sensor measurement values. For example, the processor may consider the predetermined value for the variation of the glucose measurement value is approximately 5 mg/dL or the like and the predetermined period of time is 2 hours (i.e. the variation of the glucose measurement value is not greater than the predetermined value, e.g., 5 mg/dL, over the predetermined period of time, e.g., 2 hours).

In an example, the system may receive a series of glucose measurement values that have substantially no variation or change, such as glucose measurement values that are constantly 200, 200, 200, . . . 200 for a sensor check sample period. The sensor check sample period may be a period of time or a number of operational cycles (that may be equivalent to a period of time), such as 1 hour, 2 hours, between 1 hour to 3 hours, 12 glucose measurement values, 12 operational cycles of an analyte sensor or a medicament delivery device, 6 to 24 measurement values or operational cycles, or the like).

While sensor measurement values are expected to have a variation in either a positive direction or a negative direction (e.g., increasing mg/dL or decreasing mg/dL) from one sensor measurement value to another during normal operation, when the variation of the sensor measurement values is less than a change variation threshold, the AMD algorithm may determine at 106 that the sensor measurement values are constant. The change variation threshold is an amount that the sensor measurement value is expected to vary without any disturbances to the user's diet, metabolism, or medicament delivery device. The change variation threshold may be specifically determined for the user to optimize the responsiveness of process 100. The change variation threshold enables the system to account for some inconsequential variation (i.e., where the variation would not make a difference in the operation of the AMD algorithm, or substantially no variation), the AMD algorithm may enable a tolerance such as, for example, that the successive measurement values are not the same but where a difference from one another is greater than the range of 0.0-0.75 mg/dL.

In one example threshold, this may be set as a variation of no greater than a measurement change threshold, such as, 5 mg/dL (or any value between about 1.5 mg/dL to about 10 mg/dL) over a sensor check sample period such as 90 minutes, 2 hours, 3 hours, any value between 90 minutes and 3 hours, or the like. For example, the glucose measurement value from the continuous glucose monitor (CGM) may generate a continuous reading of 200 mg/dL for 24 hours. Or even, for example, if over the 2 hours the CGM presents glucose measurement values that never deviate more or vary more than ±2.5 mg/dL from a value of 146 mg/dL, for example, for a duration of the sensor check sample period, which while appearing to be an intended result of a properly functioning AMD system, physiologically the glucose of the human body typically experiences a wider variation in glucose measurement values over time. Even when sleeping a person's glucose values may vary an appreciable amount greater than the ±2.5 mg/dL deviation or variation mentioned above. Similarly, a user's hormones may also cause noticeable deviations in a user's glucose measurement values. The lack of deviation within the glucose measurement values may be indicative of a sensor issue. The ±2.5 mg/dL deviation or variation is only an example, other variation or deviation values may be ±3.0, 0.75, 2.25, 1.75 mg/dL, or the like. In some embodiments, the measurement change threshold may be between about ±0.75 mg/dL/h to about ±3.0 mg/dL/h.

Alternatively, to accommodate the inconsequential variation, the term “constant” as used herein may additionally refer to “inconsequential variation” as within a predetermined range of variation of the sensor measurement values, such as, for example, ±0.00-0.75 mg/dL, ±0.00-0.99 mg/dL, ±0.25-0.50 mg/dL, ±1.0 mg/dL, or the like. The predetermined range of variation may also be referred to as a “change variation threshold.” Alternatively, the tolerance for the inconsequential variation may be a fixed number and not a range, such as ±0.5 mg/dL, ±0.75 mg/dL, ±1 mg/dL or the like. In the evaluation at 106, the AMD algorithm is evaluating the received sensor measurement values to confirm that there is some variation in the sensor measurement values beyond inconsequential variation.

Analyte values in the human body rarely remain in a constant state so sensor measurement values, such as glucose measurement values, which do not vary by at least some amount, e.g., outside the above examples of a predetermined range or predetermined variation, are unrealistic and almost robotic in nature. If the response at 106 is “Yes”, the two analyte readings are constant as discussed above (e.g., the two analyte readings have a difference between them that is less than a predetermined variation range), the process 100 proceeds to 118, where the AMD algorithm reverts to a default safety delivery. For example, if the received sensor measurement values are constantly 200 mg/dL for an extended period of time, the AMD algorithm may identify the sensor measurement values as “constant” and as an anomalous reading or an anomalous measurement (i.e., a “YES” output at 106), and proceed to 118.

At 118, the AMD algorithm may revert to safety basal delivery which may be a medicament delivery setting for a medicament delivery dosage amount where the setting is selected to maintain the user's glucose levels at a substantially constant level. The setting may be a default safety basal delivery setting that is selected based on predetermined data (e.g., typical glucose measurement value for time of day, insulin-on-board, or the like). In an example, prior to the reverting to a default safety basal delivery setting, the AMD algorithm executing the process 100 may be operable to generate one or more notifications and/or inquiries related to a user's activities (e.g., participating in exercise, stressful event, consuming a meal, previously consumed a meal, or the like).

After 118, the process 100 proceeds to 120, where the AMD algorithm is operable to present indication of potential sensor issue. The presented indications may be alerts to check the sensor, re-set the sensor, calibrate the sensor, inquire about sensor location or the like. After 120, the glucose error detection (GED) module 122 may be operable to repeat the process 100 upon receipt of another sensor measurement value.

Alternatively, if the response at 106 is “No”, the two analyte readings are not constant (i.e., the two analyte readings have a difference between them that is greater than a predetermined range or predetermined variation), the process 100 may proceed to continue to the evaluation of the sensor measurement value at constant change 108.

With a properly operating sensor, small incremental changes over a period of time, where a rate of change is a small variation (e.g., ±0.2 mg/dL), or the slope of the change is very gradual or consistent (nearly a linear slope) are expected. For example, a user's glucose readings typically do not vary at an absolute constant rate of change over an extended duration.

In the constant change evaluation at 108, the AMD algorithm evaluates the sensor measurement values for a constant change. “Constant change” may refer to an amount of change in sensor measurement values that is substantially the same between successive sensor measurement values or changing in a consistent manner (i.e., a change variation) that is atypical. Therefore, if a sensor measurement values exhibit a pattern of constant change that is either an increase or a decrease in the sensor measurement value of the successive readings, such patterns may be detected and indicated to the user as a potential mechanical, electrical, and/or software fault of the sensor. Such patterns may also be referred to as “consistent pattern”. In response to the potential mechanical, electrical, and/or software fault of the sensor, the AMD algorithm may revert a medicament delivery setting to a safety basal delivery setting.

When the evaluation of a constant change is performed by the AMD algorithm at 108, the received sensor measurement values are evaluated to determine whether the variation or change in the sensor measurement values is a “constant change” in one direction (i.e., increasing mg/dL where the value of the increased is a fixed variation or decreasing similarly). In an example of a change variation threshold, the change variation threshold may be set as a constant change in glucose readings per cycle that varies by no more than 1 mg/dL/min (i.e., a change in glucose of 5 mg/dL/5 min, then 6 mg/dL/5 min, then 5 mg/dL/5 min would qualify), for more than 1 hour. In some embodiments, the change variation threshold may be set as a constant change in glucose readings per cycle that varies by no more than about 0.25 mg/dL/min to about 2 mg/dL/min, more specifically between about 0.5 mg/dL/min to about 1.5 mg/dL/min, and in particular 1 mg/dL/min.

In a detailed example, at 108, the AMD algorithm may be operable to evaluate each of the received glucose measurement values with respect to one another to determine whether a change variation threshold of the glucose measurement values is satisfied. The change variation threshold may, for example, be a range from approximately 2.5 mg/dL to approximately 10 mg/dL.

In an example threshold, this may be set as a constant change in glucose readings per cycle that varies by no more than 1 mg/dL/min for more than 1 hour. This type of constant change in sensor measurement values may include a component referred to as “a change variation” that may be evaluated with respect to a threshold (i.e., a change variation threshold). In more detail, a constant change may be a change, for example, of 5 mg/dL from a previous glucose measurement value to a glucose measurement value for a first operational cycle (e.g., within 5 min), a change of 6 mg/dL for a second operational cycle (e.g., within 5 min) from the glucose measurement value of the first operational cycle, a change of 7 mg/dL for a third operational cycle (e.g., within 5 min) from the glucose measurement value of the second operational cycle, and so on, with that trend or a pattern of variation continuing for a period of time, such as a sensor check sample period as described above (i.e. a change in glucose of 5 mg/dL/5 min, then 6 mg/dL/5 min, then 5 mg/dL/5 min would exceed a change variation threshold and therefore, qualify as “constant change”). Therefore, if a set of glucose readings meets a pattern of constant change increase or decrease in readings (i.e., measurements), such patterns may be detected and indicated to the user as a potential mechanical or software fault of the sensor.

As such, if the sensor measurement value does not deviate more than a minor variation, such as the ±0.0-0.75 mg/dL for a glucose measurement value over a predetermined period of time, the process 100 may consider this a “NO” at 108, and may proceed to evaluate the glucose measurement value for changes outside physiological threshold (110).

Alternatively, the constant change may keep incrementing or decrementing. Examples of a measurement change threshold of the sensor measurement values, in the case of a glucose measurement value, may be set at 1 mg/dL, 1.5 mg/dL, 2 mg/dL, 5 mg/dL, any value between 1 mg/dL to 5 mg/dL, or the like. The “measurement change threshold” of the evaluation of constant measurement in 106 and the “change variation threshold” of the evaluation of constant change 108 are different thresholds. The measurement change threshold is a threshold value that is used to determine that a sensor measurement value has changed in comparison to an earlier sensor measurement value, while the “change variation threshold” is an amount of change between successive sensor measurement values that exceed the measurement change threshold. In the example above, successive sensor measurement values keep exceeding a measurement change threshold of 5 mg/dL per operational cycle, and in addition, each successive sensor measurement value exceeds the change variation threshold of 1 mg/dL as each successive sensor measurement value is received (e.g., 5 mg/dL, 6 mg/dL and 7 mg/dL). Such a constant change of the sensor measurement value is an indication for improper operation of the sensor. In some embodiments, the measurement change threshold may be set at between 2 mg/dL to 10 mg/dL per operational cycle, more specifically between about 3.5 mg/dL to about 7.5 mg/dL per cycle and in particular at about 5 mg/dL per cycle.

In addition, the change variation threshold may be a predetermined amount of variation of a glucose measurement value over a predetermined period of time, such as, for example, 1 hour, 1.5 hours, 2 hours, 3 hours, a predetermined period of time between 1 hour to 3 hours, or the like. In a further example, if the trend of the glucose measurement values is increasing and successive glucose measurement values also are increasing at continuously increasing step value (e.g., ±5 mg/dL, 6 mg/dL, 7 mg/dL and so on), the change variation threshold may be exceeded or violated.

At to 108, a “Yes, the sensor measurement values present constant changes” is generated when the evaluation of the comparison results indicate the change exceeds or violates the change variation threshold and the process 100 proceeds to 118.

After proceeding to 118, the process 100 may be operable to present indication of potential sensor issue 120. For example, the processor implementing the process 100 may cause an audio query, a visual query, or both, with respect to conditions that may have caused the sensor measurement value to exceed a change variation threshold.

Since the human body is not a static system, it would not respond with sensor measurement values that are constant measurement values (i.e., measurement values without any variation) as in the case of step 106 or constant or identical change in measurement values, such as those determined at step 108. As such, the process 100 generates a “NO” determination at 108 that the sensor measurement values are not changing by a constant value, the process 100 proceeds to step 110 to determine whether changes outside are a physiological threshold for a user.

At 110, the system is operable to determine whether the glucose measurement values vary more than a physiological threshold over a sensor check sample period. When evaluating the results of the comparison at 104, the AMD algorithm is operable to determine changes of the sensor measurement values that are outside a physiological threshold. For example, an output from a CGM may present changes in glucose measurement values that are extraordinary and outside the physiological threshold for any user.

In the example addressed at 110, a user's glucose measurement values may suddenly shift far beyond expected physiological thresholds of greater than 50 mg/dL/min between sensor glucose measurement values. Such a rapid increase or decrease may indicate potential software or hardware faults of the sensor, or potential cross-coupling errors of the sensor.

A cross-coupling error may occur, for example, when there are multiple users (e.g., multiple diabetics, multiple users of weight-loss drugs, multiple users of pain or cancer medications, and the like) within a household and through some human error (e.g., inadvertent entry of user data and/or sensor data from a first user into the medicament delivery system of the second user) or system error (e.g., inadvertent pairing between controllers, sensors, and/or medicament delivery systems).

In one example threshold, the potential software or hardware faults of the sensor, or potential cross-coupling errors of the sensor may be indicated as a sample-by-sample change in glucose measurement values by, for example, 100 mg/dL/5 minutes or greater. The 100 mg/dL/5 minutes or greater may be considered “a physiological threshold” that can be set to be a value such as 50, 100 or 200 mg/dL/5 minutes, or any value between about 10 mg/dL/5 minutes to about 200 mg/dL/5 minutes, more specifically between about 30 mg/dL/5 minutes to about 100 mg/dL/5 minutes, or the like, to avoid any possible outlier values or the like. In response to the processor determining this pattern in the sensor measurement values, the processor, at 110, may generate a “YES” response and the AMD algorithm may revert to safety basal delivery at 118.

Alternatively, should the AMD algorithm determine that “NO,” the changes in the sensor measurement values are outside a physiological threshold, the process 100 proceeds to compare to previous algorithm predictions 112.

At 112, the system compares the sensor measurement value 102 to previous algorithm predictions.

In an example of an evaluation for this type of error at 112, a user's glucose readings may suddenly shift far beyond possible physiological thresholds of a human. In one example of a physiological threshold, this may be indicated as a sample-by-sample change in glucose readings by 100 mg/dL/5 minutes or greater. The sample-by-sample change threshold (i.e., 100 mg) could be even larger to ensure that the value is likely not an outlier or that the user is not laying on the wearable medicament delivery device or the analyte sensor. This may indicate potential software or hardware faults of the analyte sensor, or potential cross-coupling errors of the sensor (e.g, another user's analyte sensor is inadvertently coupled to the drug delivery device).

The processor when executing the process 100 may note a pattern of increasing sensor measurement values that continue to exceed the physiological thresholds. A pattern may, for example, be an increase over a number of consecutive sensor measurement values, such as 12, or the like, an increase over a set period of time, such as 1 hour, or the like. When sensor measurement values having this pattern of shifting far beyond possible physiological thresholds are detected at 114 (i.e., a “Yes” response), the system may revert to safe delivery at 118.

After the compare to previous algorithm predictions 112 function, the process 100 may proceed to 114, where the AMD algorithm may be operable to determine if the sum of these deviations in, for example, the previous 1 hour versus 1-2 hours prior to the previous 1 hour are persistently increasing beyond the residual threshold th. While the residual threshold th is shown as being based on 2 hours, the residual threshold th could, of course, be other values, such as 1 hour, 1.5 hours, 3 hours, or any value between 1 hour to 3 hours, or the like. Similarly, the sensor measurement values that are compared at 112 may also be from the previous 1.5 hours, 2 hours, 3 hours, or the like.

If the determination is “YES” at 114, then this may indicate a potential sensor error, and the process 100 may proceed to revert AMD algorithm to default safety basal delivery 118.

If the response at 114 is “NO, the residuals are not constantly increasing,” the sensor measurement value 102 is delivered as an input to the AMD algorithm. The AMD algorithm may use the sensor measurement value 102 to predict future sensor measurement values, a dosage of medicament to be delivered either as a basal dosage or a bolus dosage.

In a first example, the revert AMD algorithm to default safety basal delivery 118 step is initiated without reviewing the insulin delivery history, the AMD algorithm

After process 100 takes the actions to revert the AMD algorithm to default safety basal delivery at 118, the process 100 may take an indicator from one of the evaluations 106, 108, 110, or 114 generated by the glucose error detection (GED) module 122, and output a notification that causes an output device to present an indication of a potential sensor issue 120.

After step 118 is initiated, the process 100 may proceed to step 120. At 120, the system is operable to present indication of potential sensor issues based on the evaluations at 106-114. The glucose error detection (GED) module 122 may be operable to cause the processor to present on a user interface (e.g., a touchscreen, a speaker, or the like) different notifications and/or inquiries. For example, in response to identification of a potential sensor issue, a notification or alert for the user to calibrate the analyte sensor, or check the software setting of the analyte sensor, or the like may be presented. Alternatively, or in addition, inquiries or alerts may be presented on the user interface. The inquires may be, for example: “Did you exercise?,” “Did you eat some carbohydrates while the analyte sensor was warming up?,” or the like. It is generally known that analyte sensors take some time, such as 30 minutes or so to warm up. The processor, in response to the presentation of the alert on a user interface device of the controller, is operable to receive an input via the user interface to acknowledge the anomalous measurement.

As described with respect to FIG. 1, the AMD algorithm may be configured to evaluate multiple error types for one or more different sensors, for example, by implementing process 100. For example, the evaluation at 106 may be considered a first check for a first error type of an analyte sensor in which the medicament delivery system determines whether there is any change (or variation), in either a positive (plus) direction or a negative (minus) direction, in sensor measurement values (e.g., glucose measurement values) that indicate anomalous measurements, while the evaluation of the sensor measurement value at constant change 108 may be a second check for a second error type that also indicates anomalous measurement. It may follow that the evaluation for changes outside the physiological threshold at 110 may be a third check for a third error type that indicates anomalous measurement. Yet another evaluation that compares past sensor measurement values in a first period to more recent sensor measurement values.

Although the example process 100 depicts a particular sequence of operations, the sequence may be altered without departing from the scope of the present disclosure. For example, some of the operations depicted may be performed in parallel or in a different sequence that does not materially affect the function of the process 100. In other examples, different components of an example device or system that implements the process 100 may perform functions at substantially the same time or in a specific sequence. Further, the process does not need to include all evaluation of the error types mentioned above, i.e., the process 100 may only comprise one of the steps 106, 108, 110, 114, or a combination of only 2 steps (i.e., 106 and 108, 106 and 110, 106 and 114, 108 and 110, 108 and 114, or 110 and 114) or only 3 steps, (without one of the steps of 106, 108, 110, 114). Finally, the process can also leave out step 118, thus a “YES” would directly lead to step 120, or step 120 can be omitted.

In another example of a possible problem may be a situation when a CGM does not respond. For example, a user may make an announcement of meal, or exercise but the CGM glucose measurement values provide no indication of changes to the user's glucose measurement values. In response, the AMD algorithm may generate an inquiry for presentation to the user, such as “a meal was announced, but CGM values do not indicate a meal has been consumed. Please confirm a meal has been consumed.” or the like.

Similarly, while most CGMs have an internal error detection process to determine a calibration error, the glucose error detection (GED) module could also execute processes to confirm a calibration error. CGM have their own calibration process. In this example, if a CGM is changed then the evaluation at 110 may not conducted as a calibration at time of changing CGM may have occurred that caused a change that exceeds the physiological threshold of a person. In some instances, when a user indicates a CGM has been changed, the process 100 may skip the evaluation of changes outside a physiological threshold (at 110).

While the foregoing examples covered some sensor measurement value discrepancies and error symptoms, other patterns of abnormal sensor behavior may also be detected and compensated accordingly. Reference to the medicament delivery system includes a processor and an AMD algorithm executed by the processor as well as other features as described with reference to later examples. Of course, other technical features may be readily apparent to one skilled in the art from the following figures, descriptions, and claims.

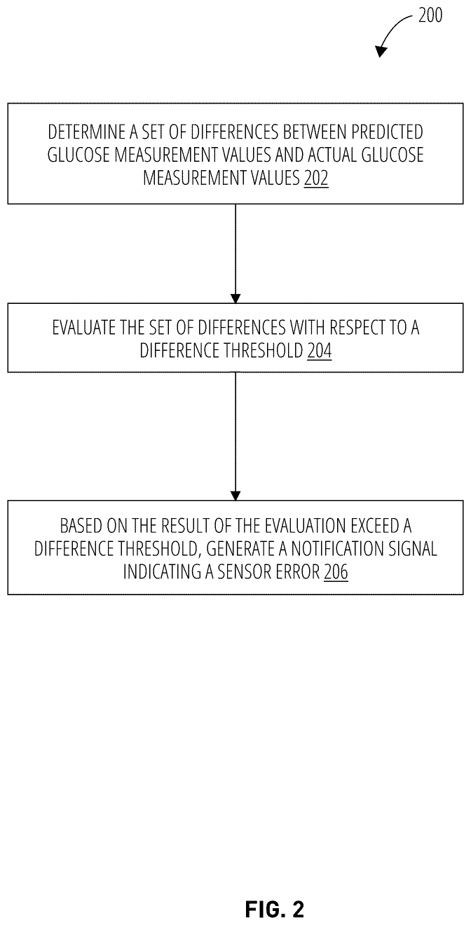

FIG. 2 illustrates another exemplary process for identifying a potential faulty analyte sensor according to the disclosed subject matter.

Prior to the executing process 200, a set of predicted sensor measurement values is generated. In this example, a set of future glucose measurement values are predicted based on prior glucose measurement values obtained from a sensor. Typically, when an analyte sensor is operating properly (i.e., outputting substantially accurate sensor measurement values, such as glucose measurement values), it is expected that the deviations between the actual sensor measurement value received at time T1 and the predicted sensor measurement value predicted earlier for time T1 are relatively consistent and within a residual threshold. However, should the analyte sensor be faulty, the deviation between the actual sensor measurement value and the predicted sensor measurement value is likely outside a residual threshold.

The process 200 illustrated in FIG. 2 is described with reference to glucose measurement values and provides a process to determine that the predicted future glucose values are the result of a sudden and consistent error in analyte sensor measurements, and the predicted future glucose values are not based on actual physiological changes of the user but instead on a faulty analyte sensor (i.e., a continuous glucose monitor (CGM)).

In an example, the internal algorithm (e.g., AMD algorithm) utilized to determine optimal insulin delivery may predict future glucose readings as part of its calculations, through an internal model. An example of such an internal model is shown in Equation 1, and predicted glucose measurements may be captured, for example, as follows:

$${G}^{′} [ k ] = {KI}^{′} [ k - 3 ] + {b}_{1} {G}^{′} [ k - 1 ] + {b}_{2} {G}^{′} [ k - 2 ] + {b}_{3} {G}^{′} [ k - 3 ] . \qquad ( Equation 1 )$$

In Equation 1, K, b₁, b₂, and b₃ may be tunable parameters that define the specific behaviors of the insulin and glucose dynamics (of a patient) within this particular model, and are used to model current G(k) and future glucose values based on previous glucose measurement value G(k−j) and insulin delivery values I(k−j). Of course, other, different models may be used so long as a substantially accurate prediction of a glucose measurement value is obtained when utilizing output from a properly operating analyte sensor.

To confirm, the analyte sensor is functioning properly, the process 200 begins at step block 202. Using a set of predicted glucose measurement value, a processor, at block 202, is operable to determine a set of differences between predicted glucose measurement values and actual glucose measurement values.

For example, the set of differences may encompass differences between actual glucose measurement values and predicted glucose measurement values for a first period of time, such as the most recent previous twelve hours (e.g., the past 1 hour (or for the past 1^{st}-12^{th} operational cycle), and differences between actual glucose measurement values and predicted glucose measurement values for a second period of time, such as the twelve hours prior to the most recent previous twelve hours (e.g., the 1 hour prior to the past 1 hour, or for the past 13^{th}-24^{th} operational cycle). It is expected that the differences are relatively consistent. Based on the set of differences, deviations of the sensor measurement values may be evaluated. In an example, the deviation in glucose measurement values, such as, for example, the difference between a present glucose measurement value and the glucose measurement value from 1 hour ago may be represented by D_{G}. The difference between the most recent predicted glucose concentrations for the k^{th} cycle for the previous 1^{st} to 12^{th} cycle are compared to the previous 13^{th} to 24^{th} previous cycles, and the differences summed square values for these predictions are calculated as D_{G}(k). In this example, the deviations in glucose measurement values (D_{G}(k)) may be calculated using Equation 2 below:

$${D}_{G} ( k ) = \sqrt{{\sum }_{j = 1}^{12} {( G ( k - j ) - {G}_{pred} ( k - j ) )}^{2}} - \sqrt{{\sum }_{j = 13}^{24} {( G ( k - j ) - {G}_{pred} ( k - j ) )}^{2}} . \qquad Equation 2$$

In Equation 2, the D_{G}(k) is the deviation between glucose measurement values (G(k−j)) and predicted glucose measurement values (Gpred(k−j)). The first term (on the left) of Equation 2 calculates a deviation of the glucose measurement values from predicted glucose measurement values for the immediately past twelve glucose measurement values (or the past 1 hour) and the second term (on the right) calculates the deviation in glucose measurement values in the past 2nd hour from the predicted glucose measurement values. The difference between the first term and the second term is D_{G}(k). The equation may also be applied using other time frames than an hourly one.

In block 204, the process 200 evaluates the set of differences with respect to a difference threshold. The difference threshold th may be calculated to account for changes in user's glucose levels over the past 24 hours by calculating the difference threshold as a percentage of the user's measured glucose levels. For example, the difference threshold th may be calculated as follows:

$$t h = 24 •12•0 \text{.01} \cdot G ( k - j ) \qquad Equation 3$$

where 24 is the number of hours in a day, 12 is the number of sensor operational cycles in 1 hour, the 0.01 is the selected percentage, and G(k−j) is the selected glucose measurement value being used.

The difference threshold th may be a tunable parameter and could have a range of values such as 250 to 2400, for example. Each of the factors of Equation 3 may be tuned for an individual user. In Equation 3, the difference threshold th may be relative to the is approximately 1% of the current glucose over a 24 hour period of sensor measurement values.

In this example, the difference between the most recent predicted glucose concentrations for the k^{th} cycle for the previous 1^{st} to 12^{th} cycle are compared to the previous 13 to 24 previous cycles, and the differences of the summed square values for these predictions are calculated as D_{G}(k). In typical embodiments, the error in the glucose predictions by this model versus actual glucose measurement values are relatively constant and/or consistent, with temporary increases in the residuals following meals, exercise, or other temporary mechanical site issues (such as pressure-induced sensor attenuation events). Accordingly, the AMD algorithm when the error in the glucose predictions versus the actual glucose measurement values may vary substantially or be inconsistent may, in a further embodiment, output inquiries related to whether the user has consumed a meal, participated in exercise, was applying pressure to (e.g., lying on) the sensor, or the like.

However, if these errors in glucose predictions demonstrate a significant, sustained change or inconsistencies in comparison to prior to previous deviations (e.g., D_{G}(k−j)), such patterns can also be detected and compensated accordingly. In an example of the AMD algorithm may detect such a sustained change or inconsistency, the AMD algorithm may calculate a sensor error utilizing a function, such as Function 1:

$${A}_{sensor error} ( k ) = \{ \begin{matrix}1 & {\sum }_{j = 1}^{24} {D}_{G} ( k - j ) - {D}_{G} ( k - j - 12 ) \geq th \\ 0 & otherwise\end{matrix} . \qquad Function 1$$

Returning to process 200, in block 206, the AMD algorithm may be operable to generate a notification signal indicating a sensor error based on the value output by Function 1. For example, if the output of Function 1 exceeds the difference threshold th (e.g., a 1 value indicates a sensor error).

Other patterns of abnormal sensor behavior may also be detected and compensated accordingly.

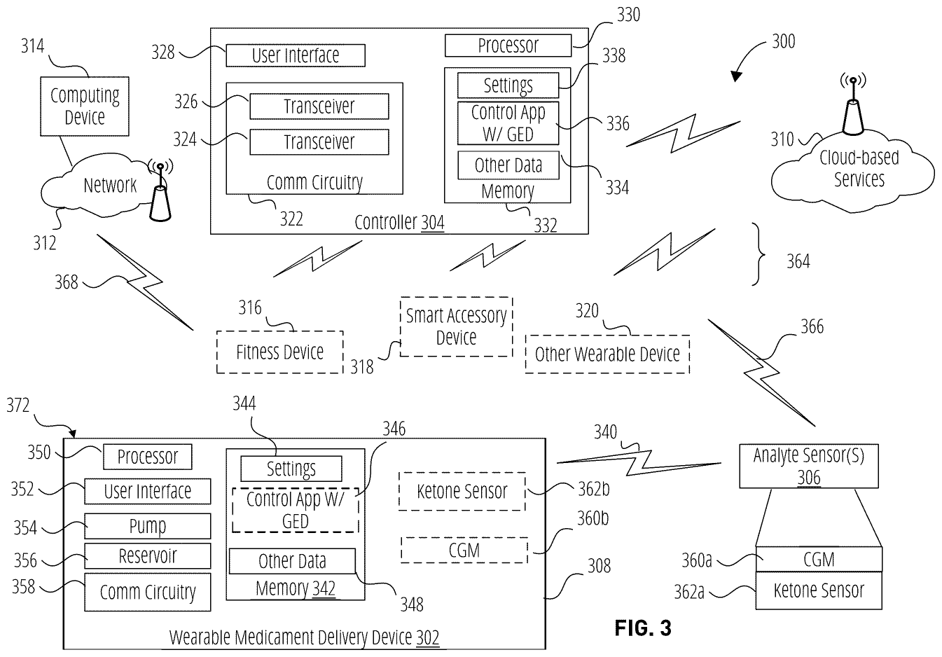

FIG. 3 illustrates an exemplary medicament system operable to implement the examples disclosed herein.

The medicament system 300 is suitable for delivering a liquid medicament, such as insulin to a user in accordance with the disclosed embodiments. The medicament system 300 may include a wearable medicament device 302, a controller 304 and an analyte sensor(s) 306.

The medicament delivery system 300 comprises a wearable medicament delivery device 302, a controller 304, an analyte sensor(s) 306, a housing(s) 308, a cloud-based services 310, a network 312, a computing device 314, a fitness device 316, a smart accessory device 318, another wearable device 320, a communication circuitry 322, a transceiver 324, a transceiver 326, a user interface 328, a processor 330, a memory 332, other data 334, a control application 336, a settings 338, a communication link 368, a memory 342, a settings 344, a control application 346, other data 348, a processor 350, a user interface 352, a pump 354, a reservoir 356, a communication circuitry 358, a continuous glucose monitor 360a, a continuous glucose monitor 360b, a ketone sensor 362a, a ketone sensor 362b, a communication links 364, a communication link 366, and a communication link 368.

The wearable medicament delivery device 302 may be a wearable device that is worn on the body of the user. The wearable medicament delivery device 302 may be directly coupled to a user (e.g., directly attached to a body part and/or skin of the user via an adhesive, or the like). In an example, a surface of the wearable medicament delivery device 302 may include an adhesive to facilitate attachment to the skin of a user.

The wearable medicament delivery device 302 may include a processor 350. The processor 350 may be implemented in hardware, software, or any combination thereof. The processor 350 may, for example, be a microprocessor, a logic circuit, a field programmable gate array (FPGA), an application specific integrated circuit (ASIC) or a microprocessor coupled to a memory. The processor 350 may maintain a date and time as well as be operable to perform other functions (e.g., calculations or the like).

The processor 350 may be operable to execute a control application 346 stored in the memory 342 that enables the processor 350 to direct operation of the wearable medicament delivery device. The control application 346 may control insulin delivery to the user per an AMD control approach as describe herein. For example, the control application 346 may be an AMD algorithm. The memory 342 may hold settings 344 for a user, such as AMD algorithm settings, such as maximum insulin delivery, insulin sensitivity settings, total daily insulin (TDI) settings, settings for the default safety basal delivery setting, parameters for Equation 2 and Function 1, and the like. In addition, the control application 346 may execute the glucose error detection (GED) module (e.g., 122 in FIG. 1) and perform the functions described with reference to the examples of FIGS. 1 and 2. The memory may also store other data 348, such as ketone values, total daily insulin values, glucose measurement values from analyte sensor(s) 306 or controller 304, insulin dosage amounts (both basal and bolus), and the like from previous minutes, hours, days, weeks, or months.

The analyte sensor(s) 306 may be operable to collect physiological condition data, such as ketone values, ketone values with a time stamp, glucose measurement values (also referred to herein as “blood glucose values” or “blood glucose”), glucose measurement values and a timestamp, and the like. In some examples, the ketone values and/or the glucose values are shared with the wearable medicament device 302, the controller 304, or both. In an additional example, the analyte sensor(s) 360a may include multiple sensors, such as a continuous glucose monitor 186 and a ketone sensor(s) 362a. In a further example, the wearable medicament device 102 may optionally include a continuous glucose monitor 360a and a ketone sensor 362a, which may be removably incorporated or fully integrated within the wearable medicament delivery device 302. For example, the continuous glucose monitor 360a and the ketone sensor 362a may be incorporated in one or more housings 372 of the wearable medicament delivery device 302.

The communication circuitry 358 of the wearable medicament delivery device 302 may, for example, be operable to communicate with the analyte sensor(s) 306 and the controller 304 as well as the devices 316, 318, and 320. The communication circuitry 358 may be operable to communicate via Bluetooth, cellular communication, near field communication (NFC), and/or other wireless protocols with the other devices 304, 306, 316, 318, and 320. While not shown, the memory 342 may include both one or more memory circuits and a combination of different types of memory circuits. For example, the memory 342 may include random access memory (RAM), read only memory (ROM), optical storage, magnetic storage, removable storage media, solid state storage or the like.

The wearable medicament delivery device 302 may include a reservoir 356. The reservoir 356 may be operable to store liquid drugs, medicaments, or therapeutic agents suitable for automated delivery, such as, insulin, glucagon-like peptide-1 (GLP-1) receptor agonist, pramlintide, glucose-dependent insulintropic polypeptide (GIP), other hormones, or co-formulations of two or more of glucagon, GLP-1, pramlintide, and insulin; as well as pain relief drugs, such as opioids or narcotics (e.g., morphine, or the like), methadone, blood pressure medicines, chemotherapy drugs, fertility drugs, or the like.

A fluid path to the user may be provided via tubing and a needle/cannula (not shown). The fluid path may, for example, include tubing coupling the wearable medicament delivery device 302 to the user (e.g., via tubing coupling a needle or cannula to the reservoir 356). The wearable medicament delivery device 302 may be operable based on control signals from the processor 350 to expel the liquid drugs, medicaments, or therapeutic agents, such as insulin, from the reservoir 356 to deliver doses of the drugs, medicaments, or therapeutic agents, such as insulin or the like, to the user via the fluid path. In a specific example, the processor 350 may be operable to cause insulin to be expelled from the reservoir 356.

There may be one or more communication links 364 with one or more devices physically separated from the wearable medicament delivery device 302 including, for example, a controller 304 of the user and/or a caregiver of the user and/or an analyte sensor(s) 306. The communication links 364 may include any wired or wireless communication link operating according to any known communications protocol or standard, such as Bluetooth®, NFC, Wi-Fi, a near-field communication standard, a cellular standard, or any other wireless protocol. The analyte sensor(s) 106 may communicate with the wearable medicament delivery device 302 via a wireless communication link 340 and/or may communicate with the controller 304 via a wireless communication link 366.

The wearable medicament delivery device 302 may also include a user interface 352, such as an integrated display device for displaying information to the user, and in some embodiments, receiving information from the user. For example, the user interface 352 may include a touchscreen and/or one or more input devices, such as buttons, knob(s), or a keyboard that enable a user to provide an input.

In addition, the processor 350 may be operable to receive data or information from the analyte sensor(s) 306 as well as other devices that may be operable to communicate with the wearable medicament delivery device 302

The wearable medicament device 302 and/or the controller 304 may interface with a network 312. The network 312 may include a local area network (LAN), a wide area network (WAN) or a combination therein. A computing device 314 may be interfaced with the network, and the computing device may communicate with the wearable medicament delivery device 302. The computing device 314 may be a healthcare provider device through which a user's controller 304 may interact to obtain information, store settings and the like. The AMD algorithm operating, as or in cooperation with, the control application 336 may present a graphical user interface on the computing device 314 enabling the input and presentation of information related to the AMD algorithm and the example techniques and processes described herein.

The medicament system 300 may include an analyte sensor(s) 306 for sensing the levels of one or more analytes of a user. The analyte levels may be used as physiological condition data and be sent to the controller 304 and/or the wearable medicament delivery device 302. The analyte sensor(s) 306 may be coupled to the user by, for example, adhesive or the like and may provide information or data on one or more medical conditions and/or physical attributes of the user. The analyte sensor(s) 306 may be a continuous glucose monitor (CGM), ketone sensor, or another type of device or sensor(s) that provides glucose measurements and/or ketone measurements. The sensor(s) 306 may be physically separate from the wearable medicament delivery device 302 or may be an integrated component thereof. The analyte sensor(s) 306 may provide the processor 330 and/or processor 350 with physiological condition data indicative of measured or detected glucose levels of the user. The information or data provided by the sensor(s) 306 may be used to modify a medicament delivery schedule and thereby cause the adjustment of medicament operations of the wearable medicament delivery device 302.

The medicament system 300 may also include the controller 304. In the depicted example, the controller 304 may include a processor 330 and a memory 332. The controller 304 may be a special purpose device, such as a dedicated personal diabetes manager (PDM) device. The controller 304 may be a programmed general-purpose device that is a portable electronic device, such as any portable electronic device, smartphone, smartwatch, fitness device, tablet or the like including, for example, a dedicated processor, such as processor, a micro-processor or the like. The controller 304 may be used to program or adjust operation of the wearable medicament delivery device 302 and/or the analyte sensor(s) 306. The processor 350 may execute processes to control the delivery of the medicament, drug, or therapeutic agent to the user for the purpose of managing a user's blood glucose and/or ketone levels. The processor 350 may also be operable to execute programming code stored in the memory 342. For example, the memory 342 may be operable to store a control application 120, such as an AMD algorithm for execution by the processor 350. The control application 346 may be responsible for controlling the wearable medicament delivery device 302, including the automated delivery of medicament, a drug, or therapeutic agent based on recommendations and instructions from the AMD algorithm, such as those recommendations and instructions described herein.

The memory 342 may store one or more applications, such as control application 346, and settings 344 for the medicament wearable medicament delivery device 302 like those described above. In addition, the memory 342 may be operable to store other data 348 and/or computer programs, such as medicament history, glucose measurement values over a period of time, total daily insulin values, sensor measurement values (e.g., glucose measurement values, ketone values, or the like), and the like. For example, the memory 342 is coupled to the processor 350 and operable to store programming instructions, such as the control application 346 and settings 344, and data, such as other data 348, related to a glucose of a user and/or data related to an amount of insulin expelled by the wearable medicament delivery device 302.

The controller 304 may include a user interface (UI) 328 for communicating with the user. The user interface 328 may include a display, such as a touchscreen, for displaying information. The touchscreen may also be used to receive input when it is a touch screen. The user interface 328 may also include input elements, such as a keyboard, button, knob or the like.

In an operational example, the controller 304 may be operable to receive glucose measurement values from an analyte sensor. In the example, the system 300 also includes a processor (e.g., 330, 350 or both) that may execute programming code, such as an AMD algorithm, and be operable to identify anomalous readings indicative of abnormal sensor behavior among the received glucose measurement values. For example, as described in earlier examples, the identified anomalous readings are based on the received glucose measurement values exceeding a measurement change threshold. The processor, in response to identifying the anomalous readings, may be operable to revert to a default safety delivery setting and/or generate an alert for presentation to a user. The processor, when identifying readings of indicative of abnormal sensor behavior, may be further operable to determine whether the glucose measurement values vary more than a physiological threshold over a sensor check sample period. The measurement change threshold may, for example, be a value representing a pattern of variability expected for a user without any disturbances to the user's diet, metabolism, or medicament delivery device. In other examples, the system may also include a variability value for the measurement change threshold is that is a predetermined value of a glucose measurement value over a predetermined period of time. In example of the variability value, the predetermined value of the glucose measurement value may be set at approximately 5 mg/dL, or the like and the predetermined period of time may be set at 2 hours, or the like. Of course, other values may be set for the predetermined value or the predetermined period of time.

The controller 304 may interface via a wireless communication link of the wireless communication link 368 with a network, such as a LAN or WAN or combination of such networks that provides one or more servers or cloud-based services 310 via communication circuitry 322. The communication circuitry 322, which may include transceivers 324 and 326, may be coupled to the processor 330. The communication circuitry 322 may be operable to transmit communication signals (e.g., command and control signals) to and receive communication signals (e.g., via transceivers 324 or 326) from the wearable medicament delivery device 302 and the analyte sensor(s) 306. In an example, the communication circuitry 322 may include a first transceiver, such as 324, that may be a Bluetooth transceiver. The first transceiver 324 may be operable to communicate with the communication circuitry 322 of the wearable medicament delivery device 302, and a second transceiver, such as 326, that may be a cellular or Wi-Fi transceiver operable to communicate via the network 312 with computing device 314 or with cloud-based services 310.

The cloud-based services 310 may be operable to receive and store user history information, such as glucose measurement values over a set period of time (e.g., days, months, years), a medicament history that includes insulin delivery amounts (both basal and bolus dosages) and insulin delivery times, types of insulin delivered, indicated meal times, glucose measurement value trends or excursions or other user-related diabetes treatment information, specific factor settings including default settings, present settings and past settings, or the like.

Other devices, like smart accessory device 318 (e.g., a smartwatch or the like), fitness device 316 and other wearable device 320 may be part of the medicament system 300. These devices may communicate with the wearable medicament wearable medicament delivery device 302 to receive information and/or issue commands to the wearable medicament wearable medicament delivery device 302. These devices 316, 318, and 320 may execute computer programming instructions to perform some the control functions otherwise performed by processor 330 or processor 350. These devices 316, 318, and 320 may include user interfaces, such as touchscreen displays for displaying enquiries or notifications, such as those described with reference to the examples of FIGS. 1-3. These devices 316, 318, and 320 may also have wireless communication connections with the analyte sensor(s) 306 to directly receive glucose level data or receive in parallel a presentation of the graphical user interface as shown in FIG. 3.

In another operational example, the controller 104 may be operable to execute programming code that causes the processor 119 of the controller 104 to perform the following functions. The processor 119 of the controller 104 may execute an AMD algorithm that is one of the control applications 120 stored in the memory 118. The processor may be operable to present notifications to an output devices of the user interface 352, such as causing the presentation of graphical user interfaces as described herein on user interface 352. The user interface 352 may be a touchscreen display controlled by the processor 350, and the user interface 352 is operable to present a graphical user interface that offers an input for responding to notifications generated by the AMD algorithm. The processor 350 may cause presentation on a user interface of a user device, a graphical user interface offering an input device that enables input of a subjective medicament need parameter (e.g., subjective to the user, or based on the user's desire). The AMD algorithm may generate instructions for the pump 354 to deliver basal medicament to the user or the like.

The processor 330 is also operable to collect physiological condition data related to the user from sensors, such as the analyte sensor(s) 306 or heart rate data, for example, from the fitness device 316 or the smart accessory device 318. In an example, the processor 330 executing the AMD algorithm may determine a dosage of medicament to be delivered based on a default safety basal delivery setting specific to the user. The processor 330 may output a control signal via one of the transceivers 324 or 326 to the wearable medicament delivery device 302. The outputted signal may cause the processor 350 to deliver command signals to the pump 354 to deliver an amount of the determined dosage of medicament in the reservoir 356 to the user based on an output of the AMD algorithm. The processor 350 may also be operable to perform calculations to update or establish settings of the AMD algorithm as discussed as herein. Modifications to the AMD algorithm settings may be stored in the memory 342, for example, as other data 348.

A wearable medicament wearable medicament delivery device 302, typically, has a lifecycle that is based on the amount of liquid drug that is stored in a reservoir 356 of the wearable medicament delivery device 302 and/or the amount of the liquid drug delivered to the user. An AMD application or algorithm may use a number of parameters, such as glucose measurement values, total daily medicament, medicament onboard, and the like, when making the determination of an amount of the liquid drug to have delivered. In an operational example, the processor 350 of the controller 304 may be operable to evaluate the effectiveness of the AMD algorithm's control of the wearable medicament delivery device 302. For example, the processor 350 may be operable to utilize historical data accessible by the processor in an evaluation of past performance of the AMD algorithm and generate recommendations for adjusting settings of the AMD algorithm accordingly as described in more detail with reference to the examples in the figures.

While the system 300 is described with reference to delivery of insulin and the use of an AMD algorithm, the system 300 may be operable to implement a medicament regimen via a medication delivery algorithm using a number of different liquid or therapeutic drugs/medicaments, such as those described above.

“AMD algorithm” refers to a component of the medicament delivery system automated medicament delivery algorithm that is executed by a processor that determines dosages of medicament to be delivered by a medicament delivery system.

“Anomalous measurement” refers to any sensor measurement value that is at least one of a constant measurement, a constant change, has a change outside a physiological threshold, or is used in a calculation where a difference threshold is exceeded.

“Anomalous reading” refers to any sensor reading that is at least one of a constant measurement, a constant change, or a change outside a physiological threshold.

“Automated medicament delivery algorithm” refers to an algorithm that evaluates physiological and user inputs to set parameters and calculate dosages of medicament for delivery.

“Change variation threshold” refers to an amount that successive sensor measurement values are expected vary for the user without any disturbances to the user's diet, metabolism, or medicament delivery device. More specifically, the term refers to an amount of change between successive sensor measurement values that exceed the measurement change threshold. Sensor measurement values that consistently exceed the change variation threshold are considered to be demonstrating constant change. Sensor measurement values that consistently exceed the change variation threshold are considered to be demonstrating constant change.

“Constant change” refers to an amount of change in sensor measurement values that is substantially the same between sensor measurement values or changing in a consistent manner that is atypical.

“Continuous glucose monitor” refers to an analyte sensor configured to measure an amount of glucose in a user's blood.

“Measurement change threshold” refers to a threshold value that is used to determine that a sensor measurement value has changed in comparison to an earlier sensor measurement value.

“Medicament” refers to liquid drugs, or therapeutic agents, such as insulin, glucagon-like peptide-1 (GLP-1) receptor agonist, pramlintide, glucose-dependent insulintropic polypeptide (GIP), other hormones, or co-formulations of two or more of glucagon, GLP-1, pramlintide, and insulin; as well as pain relief drugs, such as opioids or narcotics (e.g., morphine, or the like), methadone, blood pressure medicines, chemotherapy drugs, fertility drugs, or the like.

“Medicament delivery setting” refers to a setting of an AMD algorithm related to a dosage of medicament to be delivered by the medicament delivery system at least in a next system operational cycle, or a number of cycles for a predetermined period of time.

“Medicament delivery system” refers to a system including a controller device and a wearable medicament delivery device.

“Physiological threshold” refers to an amount of change, or a rate of change, in a user's blood glucose measurement value that is atypical, or physically impossible, to occur in the human body.

“Physiological variation threshold” refers to a variation that is more than a physiological threshold over a sensor check sample period.

“Safety basal delivery” refers to a medicament delivery setting for a medicament delivery dosage amount where the setting is selected to maintain the user's glucose levels at a substantially constant level.

“Sensor check sample period” refers to a period of time over which deviations between the measurement values output by an analyte sensor are evaluated.

“Sensor measurement value” refers to measurement values provided by an analyte sensor. The sensor measurement value is used by an AMD algorithm to determine whether a sensor is operating properly.

“Set of differences” refers to a number of differences between predicted glucose measurement values and actual glucose measurement values determined over a sensor check sample period.

“System operational cycle” refers to a series of operations in which an AMD algorithm receives a sensor measurement value from an analyte sensor and performs additional functions, such as delivery of medicament, evaluation of the received sensor measurement value, setting of AMD algorithm parameters, system status checks, and the like.

Software related implementations of the techniques described herein, such as the examples described with reference to FIGS. 1-3 may include, but are not limited to, firmware, application specific software, applet, or any other type of computer readable instructions that may be executed by one or more processors. The computer readable instructions may be provided via non-transitory computer-readable media. The various elements of the processes described with reference to the figures may be implemented in devices, apparatuses or systems that may include various hardware elements, software elements, or a combination of both. Examples of hardware elements that may be the basis for a computer processor may include structural members, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, processes, procedures, software interfaces, application program interfaces (API), instruction sets, computing code, computer code, code segments, computer code segments, words, values, symbols, or any combination thereof.

Some examples of the disclosed device or processes may be implemented, for example, using a storage medium, a computer-readable medium, or an article of manufacture which may store an instruction or a set of instructions that, if executed by a machine (i.e., a processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined solely by the preceding illustrative description.

Program aspects of the technology may be thought of as “products” or “articles of manufacture” typically in the form of executable code and/or associated data that is carried on or embodied in a type of non-transitory, machine-readable medium. Storage type media include any or all of the tangible memory of the computers, processors, or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

What is claimed is:

1. A medicament delivery system, comprising:
a receiver operable to receive glucose measurement values from an analyte sensor;
a processor operable to execute programming code and operable to:
identify an anomalous reading among the received glucose measurement values, wherein the identified anomalous reading is based on the received glucose measurement values violating a measurement change threshold indicative of abnormal sensor behavior; and
in response to identifying the anomalous reading, revert a medicament delivery setting to a default safety basal delivery setting and/or generate an alert for presentation to a user.

2. The medicament delivery system of claim 1, wherein the processor, when identifying the anomalous reading, is further operable to:
determine whether the glucose measurement values vary more than the measurement change threshold over a sensor check sample period.

3. The medicament delivery system of claim 1, wherein the measurement change threshold is a value for the received glucose measurement values expected for a user without any disturbances to the user's diet, metabolism, or medicament delivery device.

4. The medicament delivery system of claim 2, wherein the sensor check sample period is a time range between 1 to 2 hours.

5. The medicament delivery system of claim 2, wherein the measurement change threshold of the glucose measurement value is approximately 5 mg/dL.

6. The medicament delivery system of claim 2, wherein the processor is further operable to:
evaluate each of the received glucose measurement values with respect to one another to determine whether a change variation threshold of the glucose measurement values is exceeded, wherein the predetermined variation is a range from approximately 2.5 mg/dL to approximately 10 mg/dL.

7. The medicament delivery system of claim 1, wherein the measurement change threshold is a value representing a consistent pattern exhibiting a constant change of readings in the received glucose measurement values.

8. The medicament delivery system of claim 7, wherein the constant change is either a constant increase or a constant decrease in the received glucose measurement values.

9. The medicament delivery system of claim 7, wherein the constant change is a trend of the glucose measurement values of the received glucose measurement values to continually be increasing or decreasing for each operational cycle of the continuous glucose monitor for the measurement change threshold.

10. The medicament delivery system of claim 8, wherein the constant increase or decrease is categorized as a first increase or decrease in the received glucose measurement value from an immediate past glucose measurement value over a first period of time, with a subsequent second increase or decrease, greater than the first increase or decrease, in a subsequent received glucose measurement value that is an increase or a decrease in the subsequent glucose measurement value from the received glucose measurement.

11. The medicament delivery system of claim 9, wherein the measurement change threshold is 1 hour or 12 glucose measurement values.

12. The medicament delivery system of claim 1, wherein the measurement change threshold is a value representing a pattern of consistent readings exhibiting a constant increase or decrease of the readings in the received glucose measurement values by a substantially similar amount.

13. The medicament delivery system of claim 8, wherein the constant decrease is categorized as a first decrease in the received glucose measurement value from an immediate past glucose measurement value over a first period of time, with a subsequent second decrease, greater than the first decrease, in a subsequent received glucose measurement value that is a decrease in the subsequent glucose measurement value from the received glucose measurement.

14. The medicament delivery system of claim 1, wherein the measurement change threshold exceeds a physiological threshold of a user.

15. The medicament delivery system of claim 14, wherein the physiological threshold is represented by a change in glucose measurement values greater than a preselected glucose measurement value increase prior to a next received glucose measurement value.

16. The medicament delivery system of claim 1, wherein the processor is further operable to:
determine a difference between most recent predicted glucose concentrations for an operational cycle for a previous first number of operational cycles; and
compare the determined different to a determined different for a second number of operational cycles that occurred prior to the previous first number of cycles.

17. A controller, comprising:
a memory storing programming instructions; and
a processor coupled to the memory and configured to:
identify anomalous measurements indicative of abnormal sensor behavior among the received glucose measurement values, wherein the identified anomalous measurements are identified by:
comparing presently received glucose measurement values to previously received glucose measurement values,
determining whether a result of the comparing indicates that the presently receive glucose measurement values violated a measurement change threshold; and
in response to identifying the anomalous measurements, revert a medicament delivery setting to a default safety delivery setting.

18. The controller of claim 17, further comprising:
communication circuitry operable to pair with an analyte sensor, wherein the processor is further configured to:
in response to identifying the anomalous measurement, generate an alert to confirm operation of the analyte sensor that provided the presently received glucose measurement values for presentation to a user.

19. The controller of claim 17, wherein the processor is operable to:
present the alert on a user interface device of the controller, and
to receive an input acknowledging the anomalous measurement.

20. The controller of claim 17, wherein the processor is further configured to:
evaluate the presently received glucose measurement values for a constant change pattern by:
determining whether successive glucose measurement values exceed the measurement change threshold in a consistent direction over a sensor check sample period; and
determining whether an amount of change between the successive glucose measurement values exceeds a change variation threshold,
wherein the change variation threshold is set as a constant change in glucose readings per operational cycle that varies by no more than about 0.25 mg/dL/min to about 2 mg/dL/min over more than 1 hour.
